(19) United States
(12) Patent Application Publication
(10) Pub. No.: US 2026/0157607 A1
(43) Pub. Date: Jun. 11, 2026

(54) MEDICAL CONTROL DEVICE, MEDICAL CONTROL SYSTEM, MEDICAL CONTROL METHOD, AND COMPUTER-READABLE RECORDING MEDIUM

(71) Applicant: OLYMPUS MEDICAL SYSTEMS CORP., Tokyo (JP)

(72) Inventor: Masahide YAMAKI, Tokyo (JP)

(73) Assignee: OLYMPUS MEDICAL SYSTEMS CORP., Tokyo (JP)

(21) Appl. No.: 19/404,427

(22) Filed: Dec. 1, 2025

Related U.S. Application Data
(63) Continuation of application No. PCT/JP2023/041660, filed on Nov. 20, 2023.
(60) Provisional application No. 63/470,217, filed on Jun. 1, 2023.

(51) Int. Cl. A61B 1/00

(52) U.S. Cl. CPC A61B 1/00006; A61B 1/0004; A61B 1/00045

(57) ABSTRACT

A medical control device includes: a memory configured to store an association between scene information regarding an observation scene using a medical observation device, user information regarding a user who uses the medical observation device, and an operation screen; and a processor including hardware, the processor being configured to perform selective switching between a first display mode and a second display mode, when a first display mode is selected, acquire the scene information and the user information, generate an operation image based on first association information, the scene information, and the user information, and output the operation image to a display, and when the second display mode is selected, output a preset operation image to the display.

### CROSS REFERENCES TO RELATED APPLICATIONS

This application is a continuation of International Application No. PCT/JP2023/041660, filed on Nov. 20, 2023 which claims the benefit of priority of U.S. Provisional Application No. 63/470,217 filed on Jun. 1, 2023, the entire contents of which are incorporated herein by reference.

### BACKGROUND

### 1. Technical Field

The present disclosure relates to a medical control device, a medical control system, a medical control method, and a computer-readable recording medium.

### 2. Related Art

Hitherto, a medical control system including a medical observation device such as an endoscope and a medical control device that controls an operation of the medical observation device is known (see, for example, JP 2009-201593 A and JP 2021-151277 A).

In the medical control devices described in JP 2009-201593 A and JP 2021-151277 A, a touch panel that receives an operation performed by a user is provided on a front panel or the like. The touch panel is provided on a display screen of a display unit. Then, the user operates (touches) an operation image displayed on the display screen via the touch panel, thereby causing the medical control device to execute a specific function.

### SUMMARY

In some embodiments, a medical control device includes: a memory configured to store an association between scene information regarding an observation scene using a medical observation device, user information regarding a user who uses the medical observation device, and an operation screen; and a processor including hardware, the processor being configured to perform selective switching between a first display mode and a second display mode, when a first display mode is selected, acquire the scene information and the user information, generate an operation image based on first association information, the scene information, and the user information, and output the operation image to a display, and when the second display mode is selected, output a preset operation image to the display.

In some embodiments, a medical control system includes: a medical observation device configured to observe a subject; and the medical control device. The medical control device is configured to control an operation of the medical observation device.

In some embodiments, a medical control method includes: performing selective switching between a first display mode and a second display mode; when a first display mode is selected, acquiring scene information regarding an observation scene using a medical observation device and user information regarding a user who uses the medical observation device, generating an operation image based on first association information, the scene information, and the user information, and outputting the operation image to a display; and when the second display mode is selected, outputting a preset operation image to the display.

In some embodiments, provided is a non-transitory computer-readable recording medium with an executable program stored thereon. The program causes a processor of a medical control device to execute: performing selective switching between a first display mode and a second display mode; when a first display mode is selected, acquiring scene information regarding an observation scene using a medical observation device and user information regarding a user who uses the medical observation device, generating an operation image based on first association information, the scene information, and the user information, and outputting the operation image to a display; and when the second display mode is selected, outputting a preset operation image to the display.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of an endoscope system according to an embodiment;

FIG. 2 is a block diagram illustrating a configuration of a control device;

FIG. 3 is a diagram for describing an example of a medical control method;

FIG. 4 is a diagram for describing an example of the medical control method;

FIG. 5 is a diagram for describing an example of the medical control method;

FIG. 6 is a diagram for describing an example of the medical control method;

FIG. 7 is a diagram for describing an example of the medical control method;

FIG. 8 is a diagram for describing a first modified example of the embodiment;

FIG. 9 is a diagram for describing a second modified example of the embodiment;

FIG. 10 is a diagram for describing the second modified example of the embodiment;

FIG. 11 is a diagram for describing a third modified example of the embodiment; and

FIG. 12 is a diagram for describing a fourth modified example of the embodiment.

### DETAILED DESCRIPTION

Hereinafter, an embodiment for carrying out the disclosure (hereinafter, referred to as the embodiment) will be described with reference to the drawings. Note that the disclosure is not limited to the embodiment described below. Further, in the description of the drawings, the same reference signs denote the same parts.

### Schematic Configuration of Endoscope System

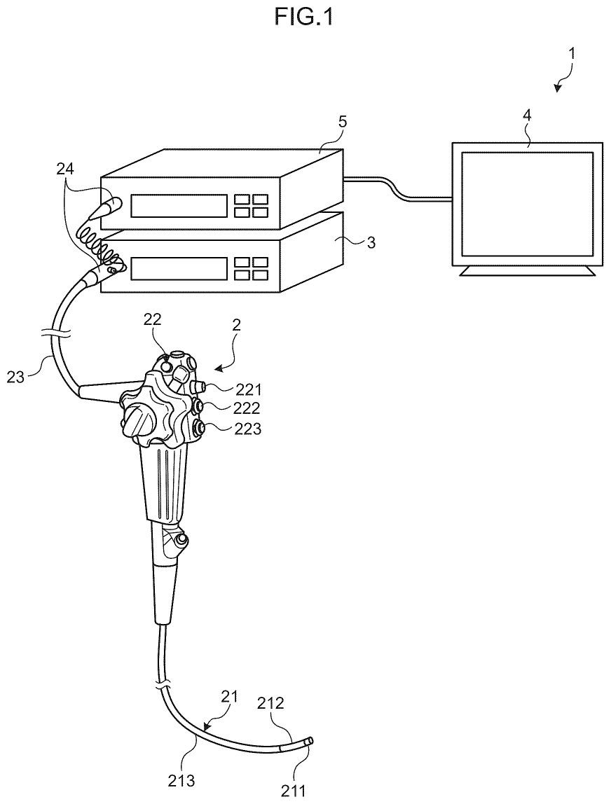

FIG. 1 is a diagram illustrating a configuration of an endoscope system 1 according to an embodiment.

The endoscope system 1 corresponds to a medical control system according to the disclosure. The endoscope system 1 is a system that is used in a medical field and observes the inside of a living body. Then, as illustrated in FIG. 1, the endoscope system 1 includes an endoscope 2, a light source device 3, a display device 4, and a control device 5.

The endoscope 2 corresponds to a medical observation device according to the disclosure.

In the present embodiment, the endoscope 2 is a so-called flexible endoscope. The endoscope 2 is partially inserted into the living body, images the inside of the living body, and outputs an image signal obtained by the imaging. Then, as illustrated in FIG. 1, the endoscope 2 includes an insertion unit 21, an operating unit 22, a universal cord 23, and a connector unit 24.

The insertion unit 21 is a portion at least a part of which has flexibility and is inserted into the living body. As illustrated in FIG. 1, the insertion unit 21 includes a distal end unit 211, a curved portion 212, and a flexible tube 213.

The distal end unit 211 is provided at a distal end of the insertion unit 21. Although not specifically illustrated, the distal end unit 211 is provided with an illumination optical system, an imaging optical system, and an imaging unit.

The illumination optical system faces one end of a light guide (not illustrated) routed in the insertion unit 21, and irradiates the inside of the living body with observation light transmitted by the light guide from the distal end of the insertion unit 21.

The imaging optical system captures return light (subject image) of the observation light which is emitted from the illumination optical system toward the inside of the living body, and forms an image on an imaging surface of an imaging element included in the imaging unit.

The imaging unit includes the imaging element such as a charge coupled device (CCD) or a complementary metal-oxide semiconductor (CMOS), captures the subject image formed by the imaging optical system, and outputs the image signal generated by the imaging. Hereinafter, the image signal is referred to as imaging data.

The curved portion 212 is coupled to a proximal end side (operating unit 22 side) of the distal end unit 211. Although not specifically illustrated, the curved portion 212 has a configuration in which a plurality of curved pieces are coupled, and is bendable.

The flexible tube 213 is coupled to a proximal end side (operating unit 22 side) of the curved portion 212, and is formed in an elongated shape having flexibility.

The operating unit 22 is connected to a proximal end portion in the insertion unit 21. Then, as illustrated in FIG. 1, the operating unit 22 is provided with a plurality of (three in the present embodiment) operation buttons 221 to 223 for receiving various operations on the endoscope 2.

The universal cord 23 is a cord extending from the operating unit 22 in a direction different from an extending direction of the insertion unit 21 and provided with the above-described light guide, a signal line for transmitting the above-described imaging data, and the like.

The connector unit 24 is provided at an end portion of the universal cord 23, and is detachably connected to the light source device 3 and the control device 5.

In the present embodiment, various endoscopes 2 of different release generations can be connected to the light source device 3 and the control device 5. Such various endoscopes 2 of different release generations are different in a type of the imaging element (CCD or CMOS), the number of pixels of the imaging element, sensitivity of the imaging element, characteristics of the illumination optical system and the imaging optical system, and the like.

The light source device 3 supplies the observation light to the other end of the above-described light guide under the control of the control device 5. Then, the observation light passes through the above-described light guide and the above-described illumination optical system, and then is emitted from the distal end of the insertion unit 21 toward the inside of the living body. Examples of the observation light include white light (normal light) which is broadband light, excitation light which is narrowband light for exciting a fluorescent agent such as indocyanine green, and narrowband light used in narrowband imaging (NBI).

The display device 4 is a liquid crystal display (LCD), an organic electro luminescence (EL) display, or the like, and displays a display image (endoscopic image) corresponding to a video signal output from the control device 5 under the control of the control device 5.

The control device 5 corresponds to a medical control device according to the disclosure. The control device 5 includes a controller such as a central processing unit (CPU) or a micro processing unit (MPU), and comprehensively controls operations of the endoscope 2 (the above-described imaging unit and the like) and the light source device 3. Note that the control device 5 is not limited to the CPU and the MPU, and may include an application specific integrated circuit (ASIC), a field-programmable gate array (FPGA), a graphics processing unit (GPU), and the like.

Note that a detailed configuration of the control device 5 will be described in “Configuration of Control Device” described later.

### Configuration of Control Device

Next, the configuration of the control device 5 will be described.

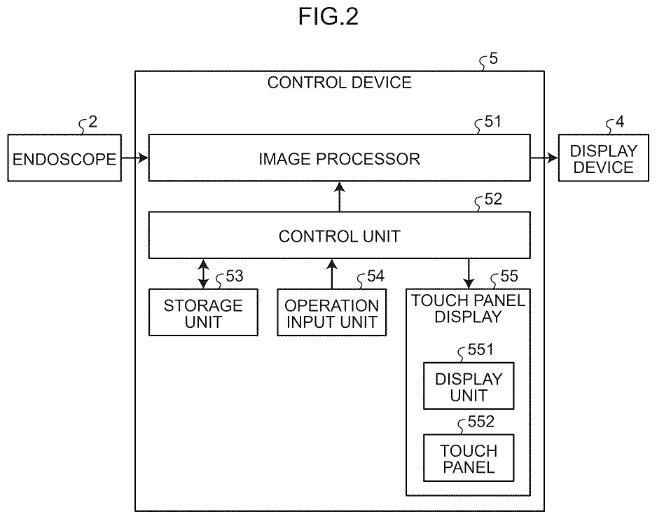

FIG. 2 is a block diagram illustrating the configuration of the control device 5.

As illustrated in FIG. 2, the control device 5 includes an image processor 51, a control unit 52, a storage unit 53, an operation input unit 54, and a touch panel display 55.

The image processor 51 performs image processing on the imaging data (RAW data) obtained by imaging using the endoscope 2, and generates the video signal corresponding to the display image to be displayed on the display device 4.

The image processing performed by the image processor 51 includes noise removal, brightness correction (gain correction), and structure enhancement. Furthermore, the image processing performed by the image processor 51 may include image processing according to the observation light (white light, excitation light, or narrowband light) emitted toward the inside of the living body. Examples of the image processing according to the observation light emitted toward the inside of the living body include optical black subtraction processing (clamp processing), white balance adjustment processing, demosaic processing, color correction matrix processing, gamma correction processing, and YC processing of converting an RGB signal into a luminance/chrominance signal (Y, Cb/Cr signals).

The control unit 52 is implemented by the controller such as the CPU or the MPU executing various programs stored in the storage unit 53, and controls the operations of the endoscope 2 (the above-described imaging unit and the like) and the light source device 3 and controls the entire operation of the control device 5. Note that the control unit 52 is not limited to the CPU and the MPU, and may be implemented by the ASIC, the FPGA, or the like. Then, the control unit 52 functions as a scene determination unit, a user information acquisition unit, and a display control unit according to the disclosure.

Note that the functions of the control unit 52 as the scene determination unit, the user information acquisition unit, and the display control unit will be described in “Operation of Endoscope System” described below.

The storage unit 53 stores a program to be executed by the control unit 52, information necessary for processing performed by the control unit 52, and the like.

Note that details of the information stored in the storage unit 53 will be described in “Operation of Endoscope System” described below.

The operation input unit 54 is implemented using an operation device such as a mouse and a keyboard, and receives a user operation. Further, the operation input unit 54 outputs, to the control unit 52, an operation signal corresponding to the user operation.

The touch panel display 55 is provided on a front panel 5A (see FIGS. 5 to 7) of the control device 5. As illustrated in FIG. 2, the touch panel display 55 includes a display unit 551 and a touch panel 552.

The display unit 551 is an LCD, an organic EL display, or the like, and displays an operation image that can be operated through the touch panel 552 under the control of the control unit 52.

The touch panel 552 is provided on a display screen of the display unit 551, and receives a user operation (touch) on the operation image displayed on the display screen. Then, the touch panel 552 outputs an operation signal corresponding to the user operation (touch) to the control unit 52.

### Operation of Endoscope System

Next, an operation of the endoscope system 1 will be described. The operation of the endoscope system 1 described below includes a medical control method according to the disclosure.

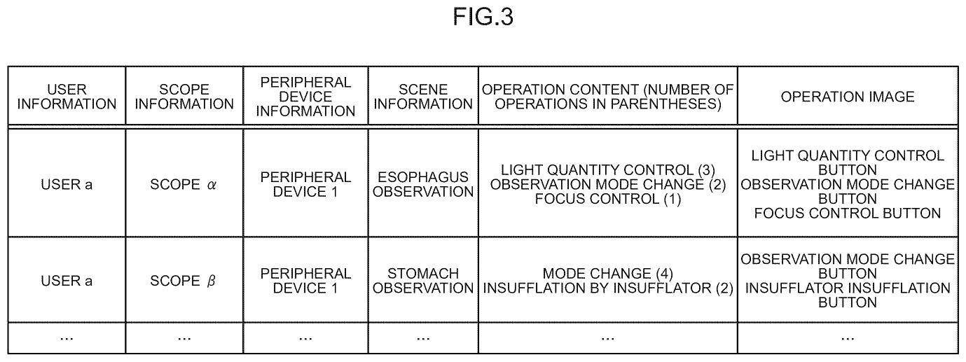

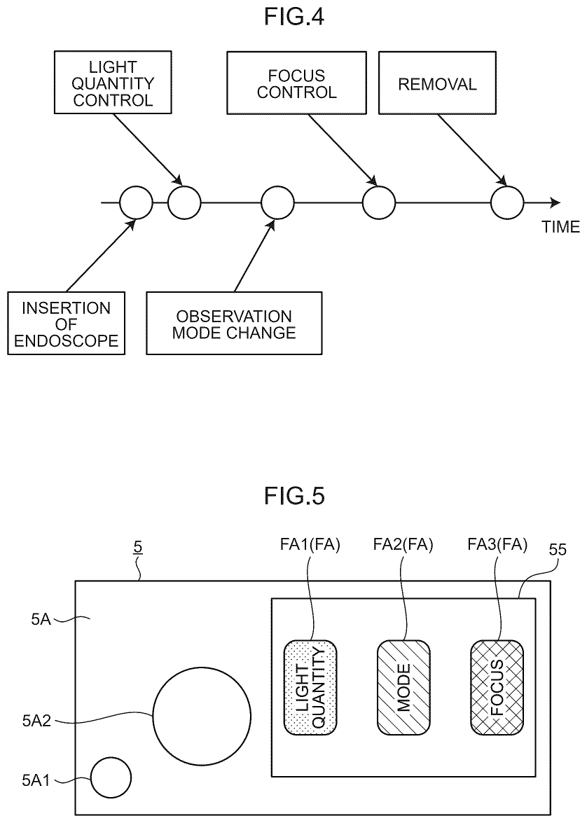

FIGS. 3 to 7 are diagrams for describing an example of the medical control method. Specifically, FIG. 3 is a diagram illustrating first association information stored in the storage unit 53. FIG. 4 is a diagram illustrating an example of a process in which the first association information is generated. FIGS. 5 to 7 are diagrams corresponding to FIG. 3, and are diagrams illustrating the operation image displayed on the touch panel display 55.

Here, the storage unit 53 stores the first association information for deriving the operation image from user information and scene information. In the present embodiment, the first association information is information for deriving the operation image from scope information and peripheral device information in addition to the user information and the scene information. The user information is information for identifying a user who uses the endoscope 2. The scene information is information regarding an observation scene of a subject using the endoscope 2. The scope information is a scope identifier (ID) of the endoscope 2 and is stored in a memory (not illustrated) provided in the endoscope 2. The peripheral device information is peripheral-device-specific information for identifying a peripheral device such as the display device 4, an image recording device, or an air/water supply unit, which is connected to the control device 5, and is stored in a memory (not illustrated) provided in the peripheral device. The scope information and the peripheral device information correspond to connected device information according to the disclosure. In the example of FIG. 3, one of the first association information is information in which “user a” which is the user information, “scope α” which is the scope information, “peripheral device 1” which is the peripheral device information, “esophagus observation” which is the scene information, “light quantity control, observation mode change, and focus control” which is an operation content, and “light quantity control button, observation mode change button, and focus control button” which are the operation images are associated with each other.

For example, in a case where the user who is an operator or an examiner has performed operations for the light quantity control, the observation mode change, and the focus control from when the endoscope 2 is inserted to when the endoscope 2 is removed as illustrated in FIG. 4 when performing a specific procedure using a specific endoscope 2 in the past, the user information for identifying the user, the scope information of the endoscope 2, the scene information (for example, esophagus observation) regarding the observation scene of the subject using the endoscope 2 at that time, and the operation images (light quantity control button, observation mode change button, and focus control button) corresponding to the operations are associated with each other and stored as the first association information in the storage unit 53.

First, the control unit 52 (scene determination unit) determines the observation scene of the subject using the endoscope 2 and calculates the scene information regarding the observation scene (scene determination step). Here, as a method of calculating the scene information, the following calculation method can be exemplified.

For example, the control unit 52 (scene determination unit) recognizes an observation target of the subject in the imaging data by image recognition (image recognition using artificial intelligence (AI)) using a trained model for scene determination, and calculates the scene information regarding the observation target. The trained model for scene determination is a trained model obtained by using the imaging data obtained by shooting various observation targets such as esophagus and stomach as teaching data and performing machine learning based on the teaching data.

Furthermore, for example, the control unit 52 (scene determination unit) estimates a distal end position of the insertion unit 21 in the subject by using the following technology. The technology is a technology of receiving magnetism generated from a magnetic coil provided in the insertion unit 21 outside the subject, and detecting a shape of the insertion unit 21 based on the received magnetism. Then, the control unit 52 recognizes the observation target of the subject based on the distal end position and calculates the scene information regarding the observation target.

Furthermore, for example, the control unit 52 (scene determination unit) calculates a distance from the distal end position of the insertion unit 21 to the observation target in the subject. Then, the control unit 52 recognizes the observation target of the subject based on the distance and calculates the scene information regarding the observation target. For example, in a case where the imaging unit of the endoscope 2 is implemented by a stereo camera, the control unit 52 calculates the distance based on the principle of triangulation by using a relative deviation amount of the same subject between images in each of captured images simultaneously captured from different viewpoints by the stereo camera. Furthermore, for example, in a case where the imaging element included in the imaging unit of the endoscope 2 is implemented by an imaging element including a phase difference pixel, the control unit 52 calculates the distance based on pixel information corresponding to the phase difference pixel.

Furthermore, for example, the control unit 52 (scene determination unit) recognizes the observation target of the subject based on a current observation state (for example, magnification observation or screening observation) using the endoscope 2, and calculates the scene information regarding the observation target.

Furthermore, the control unit 52 (user information acquisition unit) acquires the user information input by a user operation performed on the operation input unit 54 (user information acquisition step). Furthermore, the control unit 52 acquires a scope ID (scope information) from the memory (not illustrated) of the endoscope 2 connected to the control device 5. In addition, the control unit 52 acquires the peripheral device information from the memory (not illustrated) of the peripheral device connected to the control device 5.

Next, the control unit 52 (display control unit) recognizes the operation image associated with the scene information calculated as described above and the user information, the scope information, and the peripheral device information acquired as described above by referring to the first association information stored in the storage unit 53. Then, the control unit 52 (display control unit) displays the operation image on the display screen of the display unit 551 (display control step).

In the example of FIG. 5, the control unit 52 acquires “user a”, “scope α”, and “peripheral device 1” as the user information, the scope information, and the peripheral device information, respectively, and calculates “esophagus observation” as the scene information. As a result, the control unit 52 recognizes “light quantity control button, observation mode change button, and focus control button (FIG. 3)” as the operation images associated with “user a”, “scope α”, “peripheral device 1”, and “esophagus observation” with reference to the first association information. Then, the control unit 52 displays a light quantity control button FA1, an observation mode change button FA2, and a focus control button FA3, which are recognized operation images FA, on the display screen of the display unit 551 (FIG. 5).

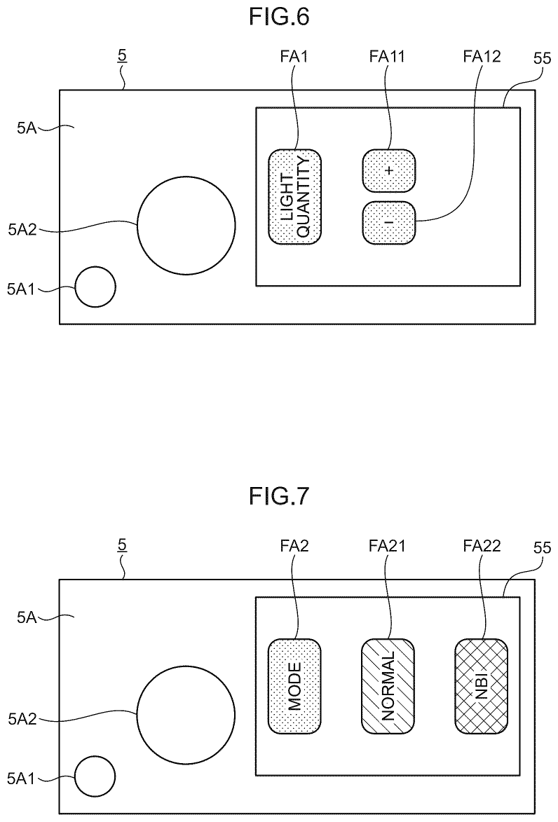

Here, the light quantity control button FA1 is an operation image for causing the control unit 52 to change a light quantity of the observation light supplied from the light source device 3. Specifically, when the light quantity control button FA1 is operated (touched) by the user, the control unit 52 displays a + button FA11 and a − button FA12, which are the operation images, on the display screen of the display unit 551 as illustrated in FIG. 6. Then, in a case where the + button FA11 is operated (touched) by the user, the control unit 52 controls the operation of the light source device 3 to increase the light quantity of the observation light supplied from the light source device 3. On the other hand, in a case where the − button FA12 is operated (touched) by the user, the control unit 52 controls the operation of the light source device 3 to decrease the light quantity of the observation light supplied from the light source device 3.

The observation mode change button FA2 is an operation image for causing the control unit 52 to change an observation mode. Specifically, when the observation mode change button FA2 is operated (touched) by the user, the control unit 52 displays a normal observation mode button FA21 and an NBI observation mode button FA22, which are the operation images, on the display screen of the display unit 551 as illustrated in FIG. 7. Then, in a case where the normal observation mode button FA21 is operated (touched) by the user, the control unit 52 changes the observation mode to the normal observation mode. The normal observation mode is a mode in which the subject is observed using white light as the observation light. On the other hand, in a case where the NBI observation mode button FA22 is operated (touched) by the user, the control unit 52 changes the observation mode to the NBI observation mode. The NBI observation mode is a mode in which the subject is observed using narrowband light as the observation light.

The focus control button FA3 is an operation image for causing the control unit 52 to adjust an image focus based on the imaging data.

According to the present embodiment described above, the following effects are obtained.

The control device 5 according to the present embodiment determines the observation scene of the subject using the endoscope 2, calculates the scene information regarding the observation scene, and acquires the user information for identifying the user who uses the endoscope 2. Then, the control device 5 derives the operation image from the calculated scene information and the acquired user information based on the first association information stored in the storage unit 53, and displays the operation image on the display screen of the display unit 551. That is, the display screen of the display unit 551 does not mixedly display the operation images that are rarely used by the user.

Therefore, with the control device 5 according to the present embodiment, it is possible to display the operation image corresponding to a function that the user desires to cause the control device 5 to execute on the display screen of the display unit 551 and to improve operability.

In particular, the first association information is information for deriving the operation image from the connected device information (the scope information and the peripheral device information) in addition to the scene information and the user information.

Therefore, when the user currently uses a connected device (the endoscope 2 and the peripheral device), the operation image operated when the user used the connected device (the endoscope 2 and the peripheral device) in the past can be displayed on the display screen of the display unit 551. Therefore, it is possible to display the operation image corresponding to the function that the user desires to cause the control device 5 to execute on the display screen of the display unit 551, and to further improve the operability.

### Other Embodiments

Although the embodiment for carrying out the disclosure has been described so far, the disclosure should not be limited only to the above-described embodiment.

Configurations of first to fifth modified examples described below may be adopted.

### FIRST MODIFIED EXAMPLE

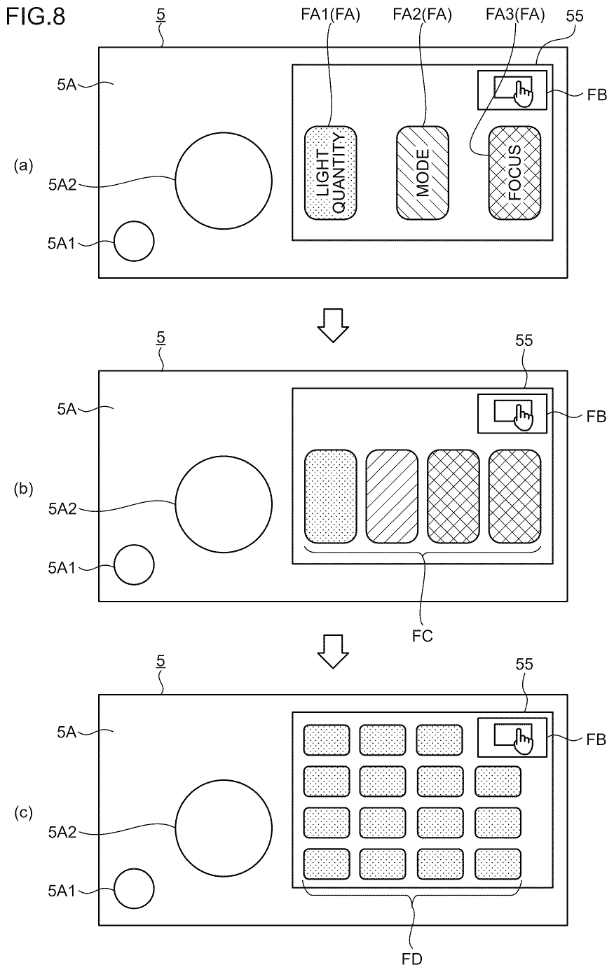

FIG. 8 is a diagram illustrating a first modified example of the embodiment. Specifically, FIG. 8 is a diagram illustrating operation images FA, FB, FC, and FD displayed on the touch panel display 55 according to the first modified example.

In the above-described embodiment, the control unit 52 (mode switching unit) may be configured to be able to switch a display mode on the display screen of the display unit 551. In the first modified example, the control unit 52 (mode switching unit) can switch the display mode among a first customized display mode ((a) of FIG. 8), a second customized display mode ((b) of FIG. 8), and an initial display mode ((c) of FIG. 8).

The first customized display mode corresponds to a first display mode. In the first customized display mode, similarly to the above-described embodiment, the control unit 52 (display control unit) displays the operation images associated with the calculated scene information and the acquired user information, scope information, and peripheral device information on the display screen of the display unit 551 by referring to the first association information stored in the storage unit 53 ((a) of FIG. 8).

The second customized display mode corresponds to a third display mode.

Here, the storage unit 53 stores the following trained model.

The trained model is a trained model generated by machine learning based on teaching data including the scene information and the operation image operated by the user on the observation scene based on the scene information using the touch panel 552. More specifically, the teaching data is data including the scene information regarding the observation scene when various users perform a specific procedure and the operation image operated by the user on the touch panel 552 in the observation scene. Then, the trained model is a learning model trained by repeatedly performing learning processing for the learning model using a plurality of pieces of teaching data. The learning model used in the learning processing is, for example, a convolutional neural network (CNN).

Note that the neural network used in the learning processing when generating the trained model is not limited to the CNN, and other neural networks may be adopted. In addition, as an algorithm of machine learning in the neural network, various known learning algorithms can be adopted. For example, a supervised learning algorithm using an error back propagation method can be adopted.

Then, in the second customized display mode, the control unit 52 (display control unit) calculates the scene information similarly to the above-described embodiment, then estimates the operation image from the scene information by using the trained model stored in the storage unit 53, and displays the operation image on the display screen of the display unit 551. In the example of (b) of FIG. 8, four operation images FC estimated using the trained model are displayed on the display screen of the display unit 551. Then, in a case where the operation image FC is operated (touched) by the user, the control unit 52 executes a function corresponding to the operation image FC.

Note that the first association information may be configured by a trained model generated by machine learning, similarly to the trained model used in the second customized display mode.

The initial display mode corresponds to a second display mode. Then, in the initial display mode, the control unit 52 (display control unit) displays a preset operation image on the display screen of the display unit 551. In the example of (c) of FIGS. 8, 15 operation images FD are displayed on the display screen of the display unit 551. Then, in a case where the operation image FD is operated (touched) by the user, the control unit 52 executes a function corresponding to the operation image FD.

Any one of the operation images FD is an operation image for troubleshooting in a case where an error or a failure such as “air cannot be supplied”, “the image cannot be recorded”, “the image is not displayed on the display device 4”, or “the image displayed on the display device 4 is distorted” occurs.

Here, as illustrated in FIG. 8, the control unit 52 (display control unit) displays the operation image FB for switching the display mode on the display screen of the display unit 551 in addition to the operation images FA to FD described above in any of the first and second customized display modes and the initial display mode. Then, in a case where the operation image FB is operated (touched) by the user, the control unit 52 switches the display mode from the current display mode to another display mode.

According to the first modified example described above, in addition to the same effect as that of the above-described embodiment, the following effects are obtained.

The control device 5 according to the first modified example can switch the display mode to one of the first and second customized display modes and the initial display mode described above.

Therefore, if the display mode is switched to the second customized display mode, it is possible to display, on the display screen of the display unit 551, the operation images operated by various users performing a specific procedure. Therefore, the user can grasp an operation required for performing the specific procedure from the display screen. That is, convenience can be improved.

Further, if the display mode is switched to the initial display mode, troubleshooting can be performed, and convenience can be improved.

### SECOND MODIFIED EXAMPLE

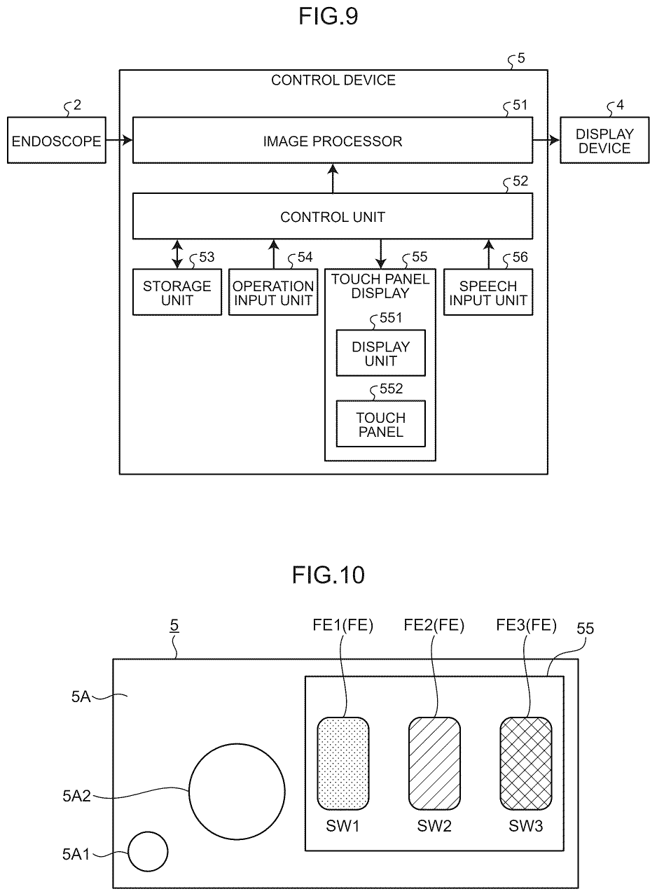

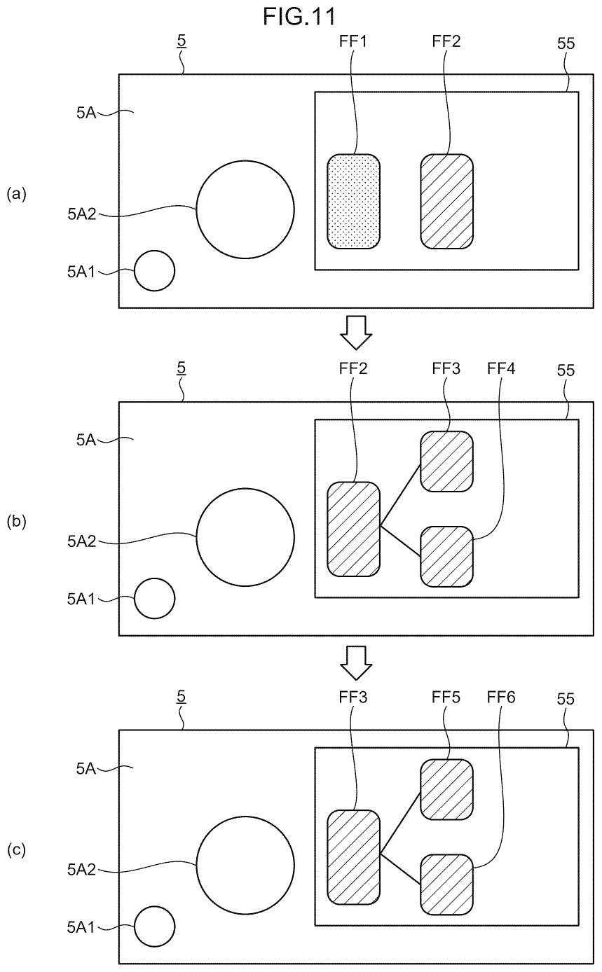

FIGS. 9 and 10 are views for describing a second modified example of the embodiment. Specifically, FIG. 9 is a block diagram illustrating a configuration of the control device 5 according to the second modified example. FIG. 10 is a diagram illustrating an operation image FE displayed on the touch panel display 55 according to the second modified example.

In the above-described embodiment, an operation image corresponding to a speech uttered by the user may be displayed on the display screen.

Here, as illustrated in FIG. 9, in the control device 5 according to the second modified example, a speech input unit 56 is added to the control device 5 described in the above-described embodiment. The speech input unit 56 includes a microphone (not illustrated) that receives a speech uttered by the user and converts the speech into an electric signal, and generates speech information by performing analog/digital (A/D) conversion or the like on the electric signal.

Furthermore, the control unit 52 according to the second modified example functions as a speech information acquisition unit and a speech analysis unit according to the disclosure, unlike the control unit 52 described in the above-described embodiment. The control unit 52 (speech information acquisition unit) acquires the speech information generated by the speech input unit 56. Furthermore, the control unit 52 (speech information analysis unit) extracts a keyword included in the speech uttered by the user by analyzing the acquired speech information.

Furthermore, the storage unit 53 according to the second modified example stores second association information for deriving the operation image from the keyword extracted by the control unit 52 (speech analysis unit). In the second modified example, the second association information is information in which the keyword and the operation image are associated with each other. Note that the second association information may be configured by a trained model generated by machine learning, similarly to the trained model used in the second customized display mode in the first modified example described above.

Then, the control unit 52 (display control unit) recognizes the operation image associated with the extracted keyword based on the second association information stored in the storage unit 53, and displays the operation image on the display screen of the display unit 551. In the example of FIG. 10, three operation images FE are displayed on the display screen of the display unit 551.

Here, the control unit 52 (display control unit) displays, on the display screen of the display unit 551, a text image of “SW1” indicating the operation button 221 of the endoscope 2 around (below in FIG. 10) an operation image FE1 among the three operation images FE. In a case where the user operates (touches) the operation image FE1 or presses the operation button 221, the control unit 52 executes a function corresponding to the operation image FE1.

In addition, the control unit 52 (display control unit) displays a text image of “SW2” indicating the operation button 222 of the endoscope 2 around (below in FIG. 10) an operation image FE2 on the display screen of the display unit 551. In a case where the user operates (touches) the operation image FE2 or presses the operation button 222, the control unit 52 executes a function corresponding to the operation image FE2.

Further, the control unit 52 (display control unit) displays a text image of “SW3” indicating the operation button 223 of the endoscope 2 around (below in FIG. 10) an operation image FE3 on the display screen. In a case where the user operates (touches) the operation image FE3 or presses the operation button 223, the control unit 52 executes a function corresponding to the operation image FE3.

According to the second modified example described above, in addition to the same effect as that of the above-described embodiment, the following effects are obtained.

The control device 5 according to the second modified example displays the operation image corresponding to the speech uttered by the user on the display screen of the display unit 551.

Therefore, in a case where the operation image desired by the user is not displayed on the display screen of the display unit 551, the user can display the desired operation image on the display screen of the display unit 551 only by speaking. That is, convenience can be improved.

In addition, since the functions corresponding to the operation images FE1 to FE3 can also be executed by the operation buttons 221 to 223 of the endoscope 2, even in a case where the insertion unit 21 is held by the hand and the hand cannot be released, the functions can be executed, and convenience can be further improved.

### THIRD MODIFIED EXAMPLE

FIG. 11 is a diagram for describing a third modified example of the embodiment. Specifically, FIG. 11 is a diagram illustrating operation images FF1 to FF6 displayed on the touch panel display 55 according to the third modified example.

In the above-described embodiment, the control unit 52 (display control unit) may update the operation image to be displayed on the display screen of the display unit 551 every time the observation scene is switched.

For example, in a case where the observation scene is a first observation scene, the control unit 52 (display control unit) recognizes the operation images FF1 and FF2 associated with the scene information regarding the first observation scene and the acquired user information, scope information, and peripheral device information by referring to the first association information stored in the storage unit 53. Then, the control unit 52 (display control unit) displays the operation images FF1 and FF2 on the display screen of the display unit 551 ((a) of FIG. 11).

Furthermore, in a case where the observation scene is switched from the first observation scene to a second observation scene, the control unit 52 (display control unit) recognizes a set of the operation images FF2 and FF3 and a set of the operation images FF2 and FF4 associated with the scene information regarding the second observation scene and the acquired user information, scope information, and peripheral device information by referring to the first association information stored in the storage unit 53. Note that the first association information stored in the storage unit 53 according to the third modified example is information in which a plurality of sets of operation images are associated with a set of the scene information, the user information, the scope information, and the peripheral device information. Then, in order to make it possible to identify the plurality of sets of operation images on the display screen of the display unit 551, the control unit 52 (display control unit) displays the operation images FF2 to FF4 in a state in which the operation image FF2 branches into two operation images FF3 and FF4 ((b) of FIG. 11).

Furthermore, in a case where the observation scene is switched from the second observation scene to a third observation scene, the control unit 52 (display control unit) recognizes a set of the operation images FF3 and FF5 and a set of the operation images FF3 and FF6 associated with the scene information regarding the third observation scene, and the acquired user information, scope information, and peripheral device information by referring to the first association information stored in the storage unit 53. Then, the control unit 52 (display control unit) displays the operation images FF3, FF5, and FF6 on the display screen of the display unit 551 in a state in which the operation image FF3 branches into two operation images FF5 and FF6 in the same manner as described above ((c) of FIG. 11).

Then, in a case where the user operates (touches) the operation images FF1 to FF6, the control unit 52 executes functions corresponding to the operation images FF1 to FF6. As the operation for executing the functions corresponding to the operation images FF1 to FF6, the operation buttons 221 to 223 and the operation input unit 54 of the endoscope 2 may be used to enable the functions, in addition to the operation (touch) on the operation images FF1 to FF6.

According to the third modified example described above, in addition to the same effect as that of the above-described embodiment, the following effects are obtained.

The control device 5 according to the third modified example updates the operation image to be displayed on the display screen of the display unit 551 every time the observation scene is switched.

Therefore, the operation images can be arranged on the display screen of the display unit 551 in an easily viewable state for the user, so that convenience can be improved.

### FOURTH MODIFIED EXAMPLE

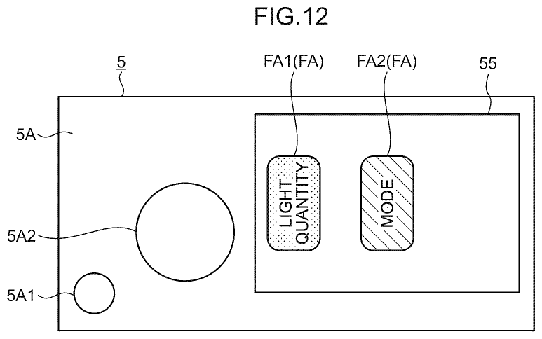

FIG. 12 is a diagram for describing a fourth modified example of the embodiment. Specifically, FIG. 12 is a diagram corresponding to FIGS. 3 to 5, and is a diagram illustrating the operation image FA displayed on the touch panel display 55 according to the fourth modified example.

In the above-described embodiment, the control unit 52 (display control unit) may be configured to display only the operation image for executing a function different from the functions executed by the operation buttons 221 to 223 of the endoscope 2 on the display screen of the display unit 551.

In the example of FIG. 12, a function corresponding to the focus control button FA3 is assigned to the operation button 221. Therefore, even in a case where the control unit 52 (display control unit) acquires “user a”, “scope α”, and “peripheral device 1” as the user information, the scope information, and the peripheral device information, respectively, calculates “esophagus observation” as the scene information, and recognizes “light quantity control button, observation mode change button, and focus control button (FIG. 3)” as the operation images associated with “user a”, “scope α”, “peripheral device 1”, and “esophagus observation” from the first association information, only the light quantity control button FA1 and the observation mode change button FA2 obtained by excluding the focus control button FA3 from the recognized operation images FA are displayed on the display screen of the display unit 551.

According to the fourth modified example described above, in addition to the same effect as that of the above-described embodiment, the following effects are obtained.

The control device 5 according to the fourth modified example displays only the operation image for executing a function different from the functions executed by the operation buttons 221 to 223 of the endoscope 2 on the display screen of the display unit 551.

Therefore, only minimum necessary operation images can be displayed on the display screen of the display unit 551, and the operation images can be arranged on the display screen in an easily viewable state for the user, so that convenience can be improved.

### FIFTH MODIFIED EXAMPLE

In the above-described embodiment and the first to fourth modified examples, a medical image processing device according to the disclosure is mounted on the endoscope system 1 in which the endoscope 2 is implemented by a flexible endoscope, but the disclosure is not limited thereto. For example, the medical control device according to the disclosure may be mounted on an endoscope system in which the endoscope 2 is a rigid endoscope. In addition, the medical control device according to the disclosure may be mounted on a medical observation system including a surgical microscope (see, for example, JP 2016-42981 A) that magnifies and observes a predetermined field-of-view region in a subject (in a living body) or on a subject surface (living body surface). In this case, the surgical microscope corresponds to the medical observation device.

With the medical control device, the medical control system, and the medical control method according to the disclosure, operability can be improved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical control device comprising:
a memory configured to store an association between scene information regarding an observation scene using a medical observation device, user information regarding a user who uses the medical observation device, and an operation screen; and
a processor comprising hardware, the processor being configured to
perform selective switching between a first display mode and a second display mode,
when a first display mode is selected, acquire the scene information and the user information, generate an operation image based on first association information, the scene information, and the user information, and output the operation image to a display, and
when the second display mode is selected, output a preset operation image to the display.

2. The medical control device according to claim 1, wherein the first association information is information for deriving the operation image operable through a touch panel provided on a display screen of the display from the scene information regarding the observation scene of a subject using the medical observation device and the user information for identifying the user who uses the medical observation device.

3. The medical control device according to claim 1, wherein the processor is configured to determine the observation scene and calculate the scene information to acquire the scene information.

4. The medical control device according to claim 1, wherein the user information is information for identifying the user.

5. The medical control device according to claim 1, wherein the processor is configured to derive the operation image from the acquired scene information and the acquired user information based on the first association information, and display the derived operation image on the display.

6. The medical control device according to claim 1, wherein the first association information is information for deriving the operation image from the scene information, the user information, and connected device information specific to a connected device connected to the medical control device.

7. The medical control device according to claim 1, wherein
the memory is further configured to store a trained model generated by machine learning based on teaching data including the scene information and the operation image operated by the user through the touch panel on the observation scene based on the scene information,
the processor is configured to
perform switching between the first display mode and a third display mode,
in the first display mode, derive the operation image from the scene information and the user information based on the first association information, and display the derived operation image on a display screen of the display, and
in the third display mode, estimate the operation image from the scene information using the trained model, and display the estimated operation image on the display screen of the display.

8. The medical control device according to claim 1, wherein
the memory is further configured to store second association information, and
the processor is configured to
acquire speech information regarding a speech uttered by the user,
extract a keyword included in the speech by analyzing the speech information,
derive the operation image from the extracted keyword based on the second association information, and
display the operation image on a display screen of the display.

9. The medical control device according to claim 1, wherein the processor is configured to update the operation image to be displayed on a display screen of the display every time the observation scene based on the scene information is switched.

10. The medical control device according to claim 1, wherein
the medical observation device is provided with an operation button configured to receive an operation performed by the user, and
the processor is configured to display only the operation image for executing a function different from a function executed by the operation button on a display screen of the display.

11. A medical control system comprising:
a medical observation device configured to observe a subject; and
the medical control device according to claim 1, the medical control device being configured to control an operation of the medical observation device.

12. A medical control method comprising:
performing selective switching between a first display mode and a second display mode;
when a first display mode is selected, acquiring scene information regarding an observation scene using a medical observation device and user information regarding a user who uses the medical observation device, generating an operation image based on first association information, the scene information, and the user information, and outputting the operation image to a display; and
when the second display mode is selected, outputting a preset operation image to the display.

13. A non-transitory computer-readable recording medium with an executable program stored thereon, the program causing a processor of a medical control device to execute:
performing selective switching between a first display mode and a second display mode;
when a first display mode is selected, acquiring scene information regarding an observation scene using a medical observation device and user information regarding a user who uses the medical observation device, generating an operation image based on first association information, the scene information, and the user information, and outputting the operation image to a display; and
when the second display mode is selected, outputting a preset operation image to the display.
